(19) United States
(12) Patent Application Publication
(10) Pub. No.: US 2008/0167384 A1
(43) Pub. Date: Jul. 10, 2008

(54) Use of Zeaxanthin For the Treatment of Diseases of the Peripheral Retina

(76) Inventors: Wolfgang Schalch, Bottmingen (CH); Regina Goralczyk, Grenzach-Wyhlen (DE)

Correspondence Address: NIXON & VANDERHYE, PC, 901 NORTH GLEBE ROAD, 11TH FLOOR, ARLINGTON, VA, 22203, US

(21) Appl. No.: 11/883,837

(22) PCT Filed: Feb. 9, 2006
(86) PCT No.: PCT/EP06/01128

§ 371 Date: Aug. 7, 2007

(30) Foreign Application Priority Data
Feb. 11, 2005 (EP) 05002877.8

(51) Int. Cl. A61K 31/045; A61P 27/02; C07C 33/02

(52) U.S. Cl. 514/739; 568/909.5

(57) ABSTRACT

Zeaxanthin is useful for the treatment or prevention of diseases of the peripheral retina, and/or for improvement of peripheral vision.

The present invention relates to a novel use of zeaxanthin. More particularly, the present invention relates to the use of zeaxanthin in the treatment or prevention of diseases associated with a degeneration and dystrophy of the peripheral retina in mammals. Such diseases include all forms of inherited or non-inherited Retinitis Pigmentosa in general, and Night Blindness (inherited, or vitamin A dependent), fundus albipunctatus, retinitis punctata albescens, Bothnia disease, Leber congenital amaurosis, Stargardt disease, Fundus flavimaculatus, Rod-cone dystrophy, Refsum disease, Bardet-Biedl (Laurence-Moon) syndrome, Best disease, Choroideremia, Gyrate-atrophy Usher syndrome, Batton disease (also known as juvenile neuronal ceroid lipofuscinosis), Bietti crystalline corneoretinal dystrophy; Wagner vitreoretinal degeneration, Stickler syndrome Retinopathy of prematurity (ROP), diabetic retinopathies, i.e. Wolfram syndrome, also known as DIDMOAD syndrome (diabetes insipidus, diabetes mellitus, optic atrophy and deafness) abetalipoproteinaemia. Zeaxanthin can also be used to treat or relief symptoms associated with chromatopsia, erythropsia, and with solar rethinopathies. Moreover, zeaxanthin can also be used to prevent and delay the dysfunction and degeneration of the peripheral retina during aging. In accordance with the present invention, zeaxanthin may furthermore be used for improvement of peripheral vision.

Retinitis Pigmentosa (RP) is the name given to a group of inherited eye diseases that affect the retina. RP causes the degeneration of photoreceptor cells in the retina. Photoreceptor cells capture and process light helping us to see. As these cells degenerate and die, patients experience progressive vision loss. There are different types of photoreceptor cells: rod cells and cone cells. Rod cells are concentrated along the outer perimeter of the retina. Rod cells help us to see images that come into our peripheral or side vision. They also help us to see in dark and dimly lit environments. Cone cells are concentrated in the macula, the centre of the retina, and allow us to see fine visual detail in the centre of our vision. Cone cells also allow us to perceive colour. Together, rods and cones are the cells responsible for converting light into electrical impulses that are transmitted to the brain where “seeing” actually occurs.

The most common feature of all forms of RP is a gradual degeneration of the rods and cones. Most forms of RP first cause the degeneration of rod cells. These forms of RP, sometimes called rod-cone dystrophy, usually begin with night blindness because patients with RP cannot adjust well to dark and dimly lit environments. As the disease progresses and more rod cells degenerate, patients lose their peripheral vision. Patients with RP often experience a ring of vision loss in their mid-periphery with small islands of vision in their very far periphery. Others report the sensation of “tunnel vision”, as though they see the world through a straw. Signs and symptoms often first appear in childhood, but severe visual problems do not usually develop until early adulthood. Many patients with RP retain a small degree of central vision throughout their life. The main risk factor is a family history of RP. The disease is affecting about 1 in 4,000 people in the U.S.

In addition to inherited forms or peripheral retinal degenerations, the photoreceptors can gradually loose their optimal functioning during aging, or upon insufficient nutrient supply to the retina, as it occurs in diseases associated with reduced blood flow to the retina, i.e. in diabetic retinopathy. Also, in other metabolic diseases (abetalipoproteinemia, dysfunction of the visual cycle of retinoids, or others listed in paragraph 1) or on suboptimal diets (vitamin A deficiency), a reduced supply of photoreceptors occurs, which can damage the cells over time and lead to impaired vision.

Thus, in one aspect, the invention relates to the use of zeaxanthin in mammals in the treatment or prevention of diseases of the peripheral retina, particularly all forms of Retinitis Pigmentosa. In a further aspect, the invention relates to the use of zeaxanthin to prevent or delay peripheral photoreceptor dysfunction associated with metabolic disease, nutritional imbalances and aging in mammals. In a further aspect of the invention, zeaxanthin is used for improvement of peripheral vision.

In still another aspect, the invention relates to the use of zeaxanthin in the manufacture of compositions for the treatment or prevention of diseases of the peripheral retina, particularly Retinitis Pigmentosa. In a further aspect of the invention, zeaxanthin is used in the manufacture of compositions for improvement of peripheral vision.

The term “peripheral vision” denotes the ability to see objects and movement outside of the direct line of vision. Peripheral vision is the work of the rods, nerve cells located largely outside the macula (the center) of the retina. The rods are also responsible for night vision and low-light vision but are insensitive to color as opposed to central vision. When peripheral vision is improved, this means that a person would be able to catch e.g. an object that is approaching him from the side much earlier and sharper, and would enable a faster reaction, i.e. to a threat. This has relevance during driving, or moving/walking in heavy traffic. It has also relevance in certain sports, i.e. soccer, basket ball, i.e. whenever objects can approach from the side, and immediate reaction is necessary. Peripheral Vision can be assessed more precisely by visual acuity tests and by visual field tests. The visual acuity test is carried out by asking the patient to read a Snellen eye chart viewed at 20 feet. The length of the room need not be 20 feet, however, as the use of mirrors to bounce the image simulates visualization at 20 feet. An individual who can resolve letters approximately one inch high at 20 feet is said to have 20/20 visual acuity. This is considered “normal” acuity. If an individual has 20/40 acuity, he or she requires an object to be at 20 feet to visualize it with the same resolution as an individual with 20/20 acuity would when the object was at 40 feet. In most states, visual acuity must be 20/40 or better with best correction in place, in at least one eye, to pass a drivers licensing test. The visual field test is done using a computerized visual field analyzer. This device systematically plots the field of vision using threshold testing, which allows the determination of retinal sensitivity in any given location. The ophthalmologist then interprets the results.

The term “composition as used herein denotes any composition that is suitable for administration to the human body, such as pharmaceutical preparations, food or beverage.

A pharmaceutical preparation in accordance with the present invention for aforesaid applications may be in any form that is conventional for oral administration, e.g. in solid form such as tablets including effervescent tablets, or soft or hard shell capsules, or in liquid form, such as solutions or suspensions, preferably oily suspension. Besides the active ingredients the pharmaceutical preparation may contain conventional pharmaceutical carrier material, additives and adjuvants, which include water, gelatin, vegetable gums, sugars, vegetable oils, polyalkylene glycols, flavoring agents, preservatives, stabilizers, emulsifying agents, buffers and the like. The medicaments may be in the form of controlled (delayed) release formulations. For the purpose of the invention the colorants as well as optional ingredients as defined earlier hereinabove may be incorporated in food or beverages, such as bakery items, e.g., cake and cookies, lemonades and fruit juices.

In solid pharmaceutical preparations, zeaxanthin is suitably present in an amount from about 0.1 mg to about 500 mg, preferably from about 1 mg to about 100 mg, especially about 6 mg to about 12 mg per dosage unit. In liquid formulations, the aforesaid ingredients are suitably present in an amount of from about 0.1 to about 5 percent by weight based upon the total weight of the composition.

A suitable daily dosage of zeaxanthin, for the purpose of the present invention is, e.g., within the range of from 0.001 mg per kg body weight to about 20 mg per kg body weight. More preferred is a daily dosage of about 0.01 to about 10 mg per kg body weight, and especially preferred is about 0.1 to 1.0 mg per kg body weight per day.

The compositions of the present invention may, additionally, contain further active ingredients for improving visual performance, such as carotenoids, e.g., lutein, mesozeaxanthin, astaxanthin, or esters thereof, or canthaxanthin, or compounds having vitamin A activity, or precursor thereof, e.g., retinol and esters thereof, such as retinyl palmitate; α- and β-carotene, β-cryptoxanthin, and lycopene. Further active ingredients for improving visual performance which may be present in the compositions of the present invention are vitamin E, vitamin C, a zinc or inorganic selenium salts such as selenophosphates, sodium selenite, sodium selenate or seleno aminoacids such as L-selenomethionine, or selenized yeast such as brewer's yeast or baker's yeast (Saccharomyces cerevisiae) containing or enriched in selenium, or bilberry extract containing approx 20 to 30 anthocyanosides, or mixtures thereof.

Lutein, mesozeaxanthin, astaxanthin, β-cryptoxanthin or esters thereof, or canthaxanthin may be present in an amount from about 0.1 mg to about 500 mg, preferably from about 1 mg to about 100 mg per dosage unit. In liquid formulations, the aforesaid ingredients are suitably present in an amount of from about 0.1 to about 5 percent by weight based upon the total weight of the composition. If vitamin E is present, its amount is suitably from about 10 to about 1000 mg per dosage unit in solid formulations and from about 0.1 to about 500 mg in liquid formulations. In liquid formulations vitamin E may serve as a carrier for other lipophilic components of the formulations in accordance with the invention and may comprise 99.9-50% percent by weight based upon the total weight of the composition. If vitamin C is present, its amount is suitably from about 10 to about 1000 mg per dosage unit. Compounds having vitamin A activity or precursors thereof may be present in amounts providing a vitamin A activity of from about 100 to about 10000 International Units per dosage unit. Zinc may be present in an amount of 1 to 100 mg (based on elementary zinc) per dosage unit. Selenium may be present in an amount of 10 to 200 microgram (based on elementary selenium) per dosage unit. Bilberry extract may be used in amounts of 50 to 150 mg (usually containing 20 to 30% anthocyanosides) per dosage unit.

The usefulness of zeaxanthin for the purposes of the present invention can be seen from the results of a trial with a duration of 6-12 months carried out with 46 subjects which were randomized to two treatment groups: Zeaxanthin, 20 mg/day (n=23), and Placebo (n=23). Macular pigment density (MPD) during this trial was monitored monthly using heterochromatic flicker photometry (HCFP) by independently measuring luminances at central and eccentric (5°=reference) locations. MPD is then calculated by subtracting the luminance at the reference (=eccentric) location from the luminance at the retinal centre.

Results: The luminances at the central as well as at the eccentric reference locations in this group both rose in parallel and to similar extent by approximately 8% during 6-12 months. The result of these parallel increases was that the used technique could not calculate an overall MPD increase, although pigment density had indeed increased but not only centrally but also eccentrically. This means that during supplementation with zeaxanthin, MPD had increased over a larger retinal area than during supplementation with lutein. Consequently, if the pre-supplementation eccentric reference luminance had been used to calculate MPD from the actual central luminances at all subsequent measuring points, an increase of about 11% would have been computed.

Conclusions: The accumulation of MPD during supplementation with zeaxanthin is not limited to the macular area at the centre of the retina but extends to eccentric retinal regions and may even reach into the far peripheral areas of the retina. Zeaxanthin thus plays a role for risk reduction of diseases occurring in the periphery of the retina such as Retinitis Pigmentosa and related diseases. Furthermore zeaxanthin may thus be regarded as important for protection of rod photoreceptors, the only photoreceptor species in the retinal periphery.

The invention is illustrated further by the Examples given below.

### EXAMPLE 1

A soft gelatin capsule may be prepared comprising the following ingredients:

| | |
|---|---|
| Zeaxanthin | 10 mg |
| Lecithin | 50 mg |
| Soy bean oil | 200 mg |

### EXAMPLE 2

A soft gelatin capsule may be prepared comprising the following ingredients:

| Ingredient | Amount per Capsule |
|---|---|
| Zeaxanthin | 6 mg |
| Vitamin E (α-d,l-tocopherol) | 200 mg |
| Vitamin C | 500 mg |
| Lecithin | 50 mg |
| Soy bean oil | 200 mg |

### EXAMPLE 3

A soft gelatin capsule may be prepared comprising the following ingredients:

| Ingredient | Amount per Capsule |
|---|---|
| Zeaxanthin | 12 mg |
| Vitamin E (α-d,l-tocopherol) | 200 mg |
| Vitamin C | 500 mg |
| Lecithin | 50 mg |
| Soy bean oil | 200 mg |

### EXAMPLE 4

A soft gelatin capsule may be prepared comprising the following ingredients:

| Ingredient | Amount per Capsule | |
|---|---|---|
| Zeaxanthin | 6 | mg |
| β-Carotene | 6 | mg |
| Vitamin E (α-d,l-tocopherol) | 200 | mg |
| Vitamin C | 500 | mg |
| Zinc (as orotate) | 7.5 | mg |
| Lecithin | 50 | mg |
| Soy bean oil | 200 | mg |

### EXAMPLE 5

A soft gelatin capsule may be prepared comprising the following ingredients:

| Ingredient | Amount per Capsule | |
|---|---|---|
| Zeaxanthin | 6 | mg |
| Vitamin E (α-d,l-tocopherol) | 200 | mg |
| Vitamin C | 500 | mg |
| Vitamin A | 1000 | Int. Units |
| Zinc (as orotate) | 7.5 | mg |
| Lecithin | 50 | mg |
| Soy bean oil | 200 | mg |

### EXAMPLE 6

A soft gelatin capsule may be prepared comprising the following ingredients:

| Ingredient | Amount per Capsule | |
|---|---|---|
| Zeaxanthin | 6 | mg |
| β-Carotene | 6 | mg |
| Vitamin E (α-d,l-tocopherol) | 200 | mg |
| Vitamin C | 500 | mg |
| Vitamin A | 1000 | Int. Units |
| Zinc (as orotate) | 7.5 | mg |
| Lecithin | 50 | mg |
| Soy bean oil | 200 | mg |

1. The use of zeaxanthin in the manufacture of a composition for oral application to the human body for the treatment or prevention of diseases of the peripheral retina selected from the group consisting of all forms of inherited or non-inherited Retinitis Pigmentosa in general, and Night Blindness (inherited, or vitamin A dependent), fundus albipunctatus, retinitis punctata albescens, Bothnia disease, Leber congenital amaurosis. Stargardt disease, Fundus flavimaculatus, Rod-cone dystrophy, Refsum disease, Bardet-Biedi (Laurence-Moon) syndrome, Best disease, Choroideremia, Gyrate-atrophy Usher syndrome, Batton disease (also known as juvenile neuronal ceroid lipofuscinosis) Bietti crystalline comeoretinal dystrophy; Wagner vitreoretinal degeneration, Stickler syndrome and abetalipo-.proteinaemia, wherein the daily dosage of zeaxanthin is within the range of from 0.001 mg per kg body weight to about 20 mg per kg body weight, preferably from about 0.01 to about 10 mg per kg body weight, and most preferably from about 0.1 to 1.0 mg per kg body weight per day.

2. The use according to claim 1 wherein the composition is for the treatment or prevention of retinitis pigmentosa.

3. The use according to claim 1 wherein the composition is a pharmaceutical composition.

4. The use according to claim 3 wherein the pharmaceutical composition contains per dosage unit an amount of about 0.1 mg to about 500 mg of zeaxanthin.

5. The use according to claim 1 wherein the composition is a food or beverage.

6. Use of zeaxanthin in the manufacture of a composition for oral application to the human body to prevent and delay the dysfunction and degeneration of the peripheral retina during aging, especially to prevent or delay peripheral photoreceptor dysfunction associated with aging.

7. A method of treating or preventing diseases of the peripheral retina and/or for improvement of peripheral vision, which comprises administering to a person in need of such treatment an effective amount zeaxanthin.

8. The method according to claim 7 for improvement of peripheral vision.

9. The method according to claim 7 wherein the disease is retinitis pigmentosa.

10. A method according to claim 7, wherein the daily dosage of zeaxanthin is within the range of from 0.001 mg per kg body weight to about 20 mg per kg body weight, preferably from about 0.01 to about 10 mg per kg body weight, and most preferably from about 0.1 to 1.0 mg per kg body weight per day.

11. The use of zeaxanthin in the manufacture of a composition for oral application to the human body for improvement of peripheral vision.

12. The use according to claim 11 wherein the composition is a pharmaceutical composition.

13. The use according to claim 12 wherein the pharmaceutical composition contains per dosage unit an amount of about 0.1 mg to about 500 mg of zeaxanthin.

14. The use according to claim 11 wherein the composition is a food or beverage.
